(19) United States
(12) Patent Application Publication
(10) Pub. No.: US 2020/0016118 A1
(43) Pub. Date: Jan. 16, 2020

(54) HDAC INHIBITOR IN COMBINATION WITH VEGF/VEGFR INTERACTION FOR CANCER THERAPY BASED ON PLATELET COUNT

(71) Applicant: 4SC AG, Planegg-Martinsried (DE)

(72) Inventors: Rolf KRAUSS, Planegg-Martinsried (DE); Takuya KIMURA, Tokyo (JP); Masatoshi KUDO, Osaka (JP)

(73) Assignees: 4SC AG, Planegg-Martinsried (DE); Yakult Honsha Co., Ltd., Tokyo (JP)

(21) Appl. No.: 15/766,197

(22) PCT Filed: Apr. 5, 2017
(86) PCT No.: PCT/EP2017/058157

§ 371 Date: Apr. 5, 2018

(51) Int. Cl. A61K 31/40; A61K 31/44; A61P 35/00

(52) U.S. Cl. CPC A61K 31/40; A61P 35/00; A61K 31/44

(57) ABSTRACT

The invention relates to methods, composition and uses for the of treatment of cancer selected from the group consisting of HCC, RCC, NSCLC, ovarian cancer, CCA, PTC and FTC in a subject, which comprise administering an HDAC inhibitor in combination with a compound inhibiting the VEGF/VEGFR interaction, wherein said subject is characterized in having a platelet count of about 140000 platelets/μl or higher.

### FIELD OF APPLICATION OF THE INVENTION

The invention relates to medical applications of an HDAC inhibitor in combination with a VEGF/VEGFR interaction inhibitor in the treatment of cancer selected from the group consisting of hepatocellular carcinoma (HCC), renal cell carcinoma (RCC), non-small cell lung cancer (NSCLC), ovarian cancer, cholangiocarcinoma (CCA), papillary thyroid cancer (PTC) and follicular thyroid cancer (FTC) in subjects with platelet count of 140000 per μl or higher.

### KNOWN TECHNICAL BACKGROUND

Hepatocellular carcinoma (HCC), also called malignant hepatoma, is the most common type of liver cancer. Most cases of HCC are a result of a viral hepatitis infection (HBC or HCV), metabolic toxins such as alcohol or aflatoxin, or conditions like hemochromatosis and alpha 1-antitrypsin deficiency or NASH.

Histone deacetylases (HDACs) are enzymes that catalyze the removal of acetyl groups from specific histone sites in particular at promotor and enhancer regions, which is an essential part of regulation of cellular gene transcription. HDACs also regulate gene expression in an indirect fashion by mediating the acetylation of non-histone proteins such as DNA-binding proteins, transcription factors, signal transducers, DNA repair and chaperon proteins (Ververis K et al., Biologics: Targets and Therapy 7: 47-60, 2013; Vitt D et al., Targeting histone acetylation. In: RSC Drug Discovery Series No. 48: Epigenetics for Drug Discovery. Editor: Nessa Carey. The Royal Society of Chemistry, 2016).

Resminostat ((E)-3-[1-(4-dimethylaminomethyl-benzenesulfonyl)-1H-pyrrol-3-yl]-N-hydroxy-acrylamide) is an orally available HDAC inhibitor histone-deacetylase (HDAC) inhibitor. HDAC inhibitors have been described to cause growth arrest with subsequent differentiation or apoptosis of tumor cells, whereas normal cells are not affected. As summarized in a review article by Marks et al. (Nature Reviews Cancer, 2001, Volume 1, page 194-202), HDAC inhibitors cause cell-cycle arrest in G1 and/or G2 phase. Growth-inhibitory effects have been documented in vitro in virtually all transformed cell types, including cell lines that arise from both hematological and epithelial tumors. The growth inhibitory cellular mechanism of the HDAC inhibitors has been described as a specific induction of expression of the cell cycle inhibitor CDKN1A (p21). Additionally, this review article summarizes the induction of growth arrest in tumor-bearing mice by HDAC inhibitors. Efficacy of HDAC inhibitors has been demonstrated in animal models of diverse cancer types such as breast, prostate, lung and stomach cancers, neuroblastoma and leukemias.

Treatment of many cancer types by HDAC inhibitors has been described in the available literature. HDAC inhibition has an effect on the expression of a number of proteins playing pivotal roles in tumor-relevant processes, such as HER2/neu, VEGF, raf-1, cyclin A and B, Bax, Bad, p53, c-myc, Caspase 3, p21 and ERα. According to a review by Villar-Garea et al. (Int. J. Cancer: 112, 171-178 (2004)) cancer is understood to be an epigenetic as well as a genetic disease and the main goal using HDAC inhibitors would be restoration of gene expression of those tumor-suppressor genes that have been transcriptionally silenced by promotor-associated histone deacetylation. Drummond et al. (Annu. Rev. Pharmacol. Toxicol. 2005. 45:495-528) review the molecular mechanism and outcome of histone and non-histone substrates in cancer cells, which are effectors of HDAC, while HDAC also facilitates the acetylation of several key proteins other than histones. According to said review, acetylation is a key posttranslational modification of many proteins responsible for regulating critical intracellular pathways, and many of these substrates are tissue/development specific (EKLF, GATA-1, ERα, MyoD), oncogenic (c-Myb), tumor-suppressing (p53), or even rather ubiquitous (TFIIE, TFIIF, TCF, HNF-4) transcription factors. Modulation of those proteins can lead to induction of cell cycle arrest, differentiation and apoptosis, all of which are desirable mechanisms for treatment of cancer. Kelly et al. (Expert Opin Invest Drugs, 11(12), 2002) provides a further review on HDAC inhibitors in general and their application in cancer therapy.

The official US NIH website http://clinicaltrials.gov lists (status: February 2016) 545 clinical trials for cancer indications treated with HDAC inhibitors, among others various forms of Leukemia (e.g. CML, CLL, AML), myelodysplastic syndrome, lymphoma including non-hodgkin's lymphoma, multiple myeloma, plasma cell neoplasm, solid tumors in general, small intestine cancer, mesothelioma, prostate, breast (male and female), lung cancer (including non-small and small cell), neuroendocrine, malignant epithelial neoplasms, pancreas, skin cancer (including melanoma), multiple myeloma, cervix, renal cell, head and neck, gastric, ovarian, liver cancer, colon, rectal, thymoma, fallopian tube, peritoneal, nasopharyngeal, vestibular schwannoma, meningioma, acoustic neuroma, neurofibromatosis type 2, thyroid, urothelial, gliomas, brain, esophagus, astrocytoma, anaplastic oligodendroglioma, giant cell glioblastoma, glioblastoma, gliosarcoma, mixed glioma, brain neoplasm, and ovarian.

The Phase IIa SHELTER study (further information is available on https://clinicaltrials.gov under the search term “shelter”) evaluated resminostat both as monotherapy and in combination with sorafenib as a second-line treatment of advanced HCC after proven radiological disease progression under first-line sorafenib therapy. The study met its primary endpoint both in the monotherapy arm and in the combination therapy. Patients receiving the resminostat/sorafenib combination therapy showed a progression-free survival rate (PFSR) after 12 weeks of 70.0% and a median PFS of 5.4 months, resulting in a median overall survival (OS) of 8.1 months.

The role of platelets in hemostasis is known and described in the literature. More recently their immunological function and role in cancer was discovered. In cancer, platelets facilitate progression and metastasis by direct and indirect interaction with the tumor cells. On the one hand platelets form aggregates with tumor cells that helps cancer cells to escape immune surveillance and prevents them from being attacked by natural killer cells (Li Int J Cancer 2016). On the other hand, platelets and factors secreted by them induce tumor growth, epithelial to mesenchymal transition and invasion (Meikle et al. Frontiers in Cell and Developmental Biology 2017; Bihari et al. APMIS 2016; Carr et al. BMC Cancer 2014; Labelle et al. Cancer Cell 2011; Carr et al. Semin Oncol 2014).

In HCC, a higher platelet count or other factors calculated therewith—like e.g. the platelet to lymphocyte ratio (PLR), alkaline phosphatese-to-platelet ratio index (APPRI), aspartate aminotransferase-to-platelet ratio index (APRI)—has been linked to worse prognosis and survival and/or a higher risk of recurrence after resection (Amano et al. J. Gastrointest Surg 2011; Tian et al. Eur Rev Med Pharmacol Sci 2016; Xia et al. World Journal of Surgical Oncology 2015; Xue et al. Tumor Biol 2015; Pang et al. World Journal Gastroenterology 2015; Yu et al. Medicine 2016; Hagiwara et al. J. Gasteroenterol 2006;). HCC patients with an underlying cirrhosis often have lower platelet counts while patients with larger tumors have elevated platelet counts.

As demonstrated by Morimoto et al. (Hepatology Research 2014) HCC patients with a higher platelet count are at risk for extrahepatic metastasis (EHM), EHM was linked by itself to a more advanced tumor stage and lower survival (Yilmaz et al. Biochem Anal Biochem 2016).

Platelet factors can even antagonize action of VEGFR inhibitors, such as sorafenib or regorafenib (D'Alessandro et al. BMC cancer 2014). Patients treated with sorafenib with a platelet count lower or equal to 150000 had an improved overall survival compared to those with a platelet count >150000 (Arizumi et al. Digestive Disease 2015—Note: This publication at first glance seems to describe the opposite.

However, it is apparent that in table 2, the “<” and “>” characters were mixed up; this can for instance be confirmed by regarding the values given for the known tumor progression markers AFP, DCP and Alpha-fetoprotein L3 in Table 2, an elevated level of which is known to indicate more advanced tumor stage, and thus decreased overall survival, respectively—this was confirmed orally by the author of the publication and will be subject of a corrigendum).

WO 2005/087724 A2 describes certain N-sulphonylpyrrole derivatives, which are described to be used in the pharmaceutical industry for the production of pharmaceutical compositions.

WO 2007/39404 A1 describes novel N-sulphonylpyrrole derivatives and certain salts of these N-sulphonylpyrrole derivatives, which are described to be used in the pharmaceutical industry for the production of pharmaceutical compositions.

WO 2009/112529 A1 describes a specific production method of N-sulphonylpyrrole derivatives and salts thereof, which are described to be used in the pharmaceutical industry for the production of pharmaceutical compositions.

### BRIEF DESCRIPTION OF THE FIGURES

FIG. 1 shows the probability of survival of patients treated with sorafenib monotherapy based on platelet count; cut off at median i.e. 151×10³/μL. As demonstrated, the probability of survival of the patients with a higher platelet count (≥median (151×10³/μL) is much smaller than for those with a lower platelet count (<median (151×10³/μL). While the patients with a platelet level below median had a median overall survival (mOS) of 17.5 months, patients with a platelet level above median (i.e. 151×10³/μL) had a mOS of only 5.1 months.

FIG. 2 shows the probability of survival of patients with a platelet count of ≥151×10³ μL at baseline treated either with sorafenib alone or with the combination of resminostat and sorafenib. By adding resminostat to the standard sorafenib treatment the median overall survival of patients was substantially increased from 5.1 months (sorafenib only) to 13.7 months (resminostat and sorafenib in combination).

FIG. 3 shows the hazard ratios in terms of overall survival of treatment with the combination of resminostat and sorafenib (“comb”) versus treatment with sorafenib alone (“mono”). In patients with a platelet count of 140×10³/μL and above hazard ratios of 0.8 were observed indicating that these patients profit from the combination therapy, compared with sorafenib monotherapy

### DESCRIPTION OF THE INVENTION

It has now been found that unexpectedly that subjects having certain types of cancer, wherein the platelet count in a blood sample obtainable from said subject is about 140000 per μL or higher, benefit particularly from treatment with a combination of an HDAC inhibitor and a compound inhibiting the VEGF/VEGFR interaction.

Certain embodiments of the present invention are listed in the following items:
1. A method of treatment of a cancer selected from the group consisting of HCC, RCC, NSCLC, ovarian cancer, CCA, PTC and FTC, said method comprising administering to a subject in need thereof an HDAC inhibitor in combination with a compound inhibiting the VEGF/VEGFR interaction, wherein said subject is characterized in having a platelet count of about 140000 platelets/μl or higher.
2. A composition comprising an HDAC inhibitor for treating cancer selected from the group consisting of HCC, RCC, NSCLC, ovarian cancer, CCA, PTC and FTC in a subject, wherein said composition is to be administered in combination with a compound inhibiting the VEGF/VEGFR interaction, and wherein said subject is characterized in having a platelet count of about 140000 platelets/μl or higher.
3. Use of an HDAC inhibitor for the manufacture of a medicament for use in the treatment of selected from the group consisting of HCC, RCC, NSCLC, ovarian cancer, CCA, PTC and FTC in combination with a compound inhibiting the VEGF/VEGFR interaction in a subject, wherein said subject is characterized in having a platelet count of about 140000 platelets/μl or higher.
4. The method according to item 1, the composition according to item 2 or the use according to item 3, wherein said HDAC inhibitor is resminostat or a salt or solvate thereof.
5. The method, composition or use according to item 4, wherein said salt of resminostat is resminostat mesylate salt.
6. The method, composition or use according to any one of the preceding items, wherein said cancer is HCC.
7. The method, composition or use according to any one of the preceding items, wherein said compound inhibiting the VEGF/VEGFR interaction is sorafenib.
8. The method, composition or use according to any one of the preceding items, the platelet count in a blood sample obtainable from said subject is about 150000 platelets/μl or higher.

As used herein, the term “platelet” is used synonymously with “thrombocyte”.

In particular embodiments of the present invention, the subject is a human subject.

In certain particular embodiments of the present invention, said subject has a platelet count of about 120000 per μL or higher, about 130000 per μL or higher, about 140000 per μL or higher, or about 150000 per μL or higher, in a particular embodiment about 151000 per μL or higher.

In certain embodiments, the method, use or combination of the present invention may involve determining said subject's platelet count, and if the platelet count in said subject is about 140000 per μL or higher, or about 150000 per μL or higher, in a particular embodiment about 151000 per μL or higher), identifying said subject as eligible for treatment with an effective amount of an HDAC inhibitor in combination with a compound inhibiting the VEGF/VEGFR interaction. Consequently, if the platelet count is lower than the aforementioned thresholds, the subject is identified as not eligible for said treatment.

In certain embodiments, the platelet count can be determined in a blood sample obtainable from said subject, particularly in a whole blood sample obtainable from said subject.

In the present invention, the blood platelet count is determined to stratify subjects into those who are eligible for treatment and those who are not eligible for treatment. Thus, the present invention is also directed to a method of stratifying a subject suffering from a cancer selected from the group consisting of HCC, RCC, NSCLC, ovarian cancer, CCA, PTC and FTC, wherein said method is characterized in
1) determining the platelet count in said subject and
2) if the platelet count in said subject is about 140000 per μL or higher, or about 150000 per μL or higher, in a particular embodiment about 151000 per μL or higher), identifying said subject as eligible for treatment with an HDAC inhibitor in combination with a compound inhibiting the VEGF/VEGFR interaction. Consequently, if the platelet count is lower than the aforementioned thresholds, the subject is identified as not eligible for said treatment.

It is apparent that the blood platelet count typically is determined prior to the treatment with an HDAC inhibitor in combination with a compound inhibiting the VEGF/VEGFR interaction.

The VEGFR inhibitor Sorafenib has received marketing approved for the treatment of advanced hepatocellular carcinoma, as well as for advanced renal cell carcinoma (RCC), and refractory differentiated thyroid carcinoma (European Medicines Agency: Nexavar (Sorafenib): Summary of Product Characteristics. Http://Www.Ema.Europa.Eu (2014)).

Platelets and platelet count play a major role in driving epithelial-mesenchymal transition (EMT) which is a critical pathway for a more spread and metastasized disease and is related to a generally worse prognosis, which is observable in HCC, RCC and thyroid carcinoma (Labelle, M. & Begum, S, Cancer Cell 20, 576-590 (2013)); Gu, L. et al., PLoS One 10, 2-13 (2015); Ekpe-Adewuyi E et al., Oncotarget 7, 83684-83700 (2016)). A similar role of the platelets has also been demonstrated in other tumor indications like ovarian cancer (Bottsford-Miller et al. Clin Cancer Res. 2015 Feb. 1; 21(3): 602-610), non-small cell lung cancer (NSCLC) (Zhao et al. Int J Cancer 2016, 139, 1, 164-170); N Inagaki et al. Lung Cancer 83 (1), 97-101; Zhang et al. Nature Scientific Reports 2016, Article number: 22618 (2016); doi:10.1038/srep22618), cholangiocarcinoma (Watanabe A et al., Ann Surg Oncol 2016, 23, 886-891) and renal cell carcinoma (Prokopowicz et al. BioMed Research International 2016, Article ID 8687575).

Herein, it has been found that the combination of an HDAC inhibitor with an inhibitor of VEGF/VEGFR interaction in HCC patients with a platelet count above a certain level as disclosed herein shows an unexpected effect over monotherapy with an inhibitor of VEGF/VEGFR interaction. In view of the above similarities between HCC, RCC, NSCLC, ovarian cancer, CCA, PTC and FTC and the biology and phenotype of these cancers, it is plausible that the effect of the combination of HDAC inhibitor and VEGF/VEGFR interaction inhibitor is present in all the aforementioned cancer types.

In certain embodiments of the present invention, the cancer is selected from the group consisting of HCC, RCC, CCA, PTC and FTC, in other certain embodiments from HCC and RCC.

In certain embodiments of the present invention, the compound inhibiting the VEGF/VEGFR interaction is a VEGFR inhibitor, such as a VEGFR1, VEGFR2 or VEGFR3 inhibitor, particularly a VEGFR2 inhibitor, or a VEGF inhibitor.

In the present invention, the HDAC inhibitor and the compound inhibiting the VEGF/VEGFR interaction are typically to be administered in therapeutically effective amounts.

The HDAC inhibitor and the compound inhibiting the VEGF/VEGFR interaction are each meant to be inclusive of their respective salts, solvates and hydrates.

In certain embodiments a VEGFR inhibitor is a compound for which an IC₅₀ of 1 μM or lower, or 500 nM or lower, or 250 nM or lower, or 100 nM or lower, or 50 nM or lower, or 25 nM or lower or can be determined for one or more VEGRF enzymes selected from the group consisting of VEGFR1, VEGFR2 and VEFR3, particularly VEGFR2, in an in vitro assay. Such in vitro assay can for instance be the following assay:

1. Prepare Peptide substrate, poly[Glu:Tyr] (4:1), 0.2 mg/ml, in freshly prepared Base Reaction Buffer (20 mM Hepes (pH 7.5), 10 mM MgCl₂, 1 mM EGTA, 0.02% Brij35, 0.02 mg/ml BSA, 0.1 mM Na₃VO₄, 2 mM DTT, 1% DMSO);

2. Add VEGFR kinase to the above solution and mix;

3. Add compound (in different predefined final concentrations for each respective data point) to be analyzed, dissolved in DMSO to the above solution, incubate for 20 minutes at room temperature (25° C.);

4. Add 100 mixture of ATP and ³³P-ATP to a final specific activity of 10 μCi/μl to the above solution;

5. Incubate kinase reaction for 2 hours at room temperature;

6. Spot onto P81 ion exchange paper filter (Reaction Biology, PA, USA);

7. Remove unbound phosphate by washing the filters in 0.75% phosphoric acid;

8. After subtraction of the background, which is derived from control reactions containing inactive enzyme) express kinase activity data as percent remaining kinase activity in the test samples compared to vehicle (DMSO) reactions;

9. Obtain IC₅₀ values and curve fits, e.g. with Graph Pad Software Prism®.

In certain embodiments of the present invention, the compound inhibiting the VEGF/VEGFR interaction is selected from the group consisting of 1,2,3-carboxyamido-triazole; 2-methoxyestradiol; A-006; AAV2.Flt23k (University of Utah); AAVrh10.BevMab (Weill Medical College of Cornell University); ABS-393 (Aparna Biosciences Corp); ABT-165 (AbbVie Inc); ACU-6151 (Acucela Inc; EyeMedics LLC); AD-051.4 (Adamed Group; Pullan Consulting); ADVM-022 (Adverum Biotechnologies Inc); afatinib; AFG-2 (Affilogic Laboratories); aflibercept; AG-321 (Duquesne University; National Cancer Institute; University of Oklahoma); AK-109 (Akeso Biopharma Inc); AL-2846 (Advenchen Laboratories LLC; Chia Tai Tianqing Pharmaceutical Group Co Ltd); AL-8326 (Advenchen Laboratories LLC); albendazole; alferminogene tadenovec; altiratinib; AMC-303 (Amcure GmbH); AMD-AAV7 (REGENXBIO Inc); AN-019; anlotinib; apatinib; apratoxin S8; APX-004 (Apexigen Inc; Hengkang Medical Group Co Ltd); AVA-101 (Adverum Biotechnologies Inc; Lions Eye Institute of Western Australia Inc; University of Western Australia); AVA-201 (Adverum Biotechnologies Inc); axitinib; AXT-107 (Asclepix Therapeutics LLC); BAT-1706 (Bio-Thera Solutions Ltd); bevacizumab; BI-836880 (Boehringer Ingelheim); BMS-817378 (Bristol-Myers Squibb Co; Simcere Pharmaceutical Group); BNC-420 (Bionomics Ltd; Cancer Therapeutics CRC Pty Ltd); BR-55 (Bracco Research SA); brivanib; BS-A3 (Chong Kun Dang Pharmaceutical Corp); BX-0510 (BioXpress Therapeutics SA); BX-2314 (BioXpress Therapeutics SA); cabozantinib; cediranib; cetuximab; CFAK-C10 (CureFAKtor Pharmaceuticals LLC); CFAK-C4 (CureFAKtor Pharmaceuticals LLC); CFAK-C9A (CureFAKtor Pharmaceuticals LLC); CG′806 (CrystalGenomics); CG-026806 (CrystalGenomics); CG-026828 (CrystalGenomics); CG-806(CrystalGenomics); CHS-3351 (Coherus BioSciences Inc); cobimetinib; conivaptan; COT601-M06.1 (Critical Outcome Technologies Inc); COT604-M06.2 (Critical Outcome Technologies Inc); crizotinib; CS-2164 (Shenzhen Chipscreen Biosciences Ltd); CS-3158 (Shenzhen Chipscreen Biosciences Ltd); CTx-0294886 (Cancer Therapeutics CRC Pty Ltd); CTx-294886 (Cancer Therapeutics CRC Pty Ltd); CX-1003 (Konruns Pharmaceutical Co Ltd); CX-1026 (Konruns Pharmaceutical Co Ltd); DA-3131 (Dong-A ST Co Ltd); dacomitinib; dalantercept; DB-83-LM4 (Novartis Institutes for BioMedical Research Inc); UF-61-QB443 (Novartis Institutes for BioMedical Research Inc); DE-120 (Santen Inc); Debio-1144 (Ascepion Pharmaceuticals Inc; Debiopharm SA); DIG-KT (3SBio Inc; PharmAbcine; Triphase Accelerator Corp); E-10C (Guangzhou Doublle Bioproducts Co Ltd); EF-24 (Emory University); EF-31 (Emory University); L-2395 (Emory University); UBS-109 (Emory University); EGEN-002 (Celsion Corp); EGEN-RNA-002; GEN-2 (Celsion Corp); ENMD-2076 (CASI Pharmaceuticals Inc); ENV-1305 (Envisia Therapeutics); Eos-003 (EOS BioSciences Inc); Eos-003 (EOS BioSciences Inc); erlotinib; EYS-609 (Eyevensys); F-0001 (Shanghai Fudan-Zhangjiang Bio-Pharmaceutical Co Ltd); F-16 (Lombardi Comprehensive Cancer Center at Georgetown University Hospital; Nova Southeastern University (NSU)); famitinib; fenretinide; ficlatuzumab; FP-1039 (Five Prime Therapeutics Inc); fruquintinib; FYB-201 (Bioeq GmbH; Formycon GmbH; Santo Holding AG); galunisertib; gefitinib; GFB-204 (Yale University); ginsenoside Rg3; glesatinib; GNR-011 (Affitech A/S; International Biotechnology Center Generium LLC); HL-217 (Hanlim Pharmaceutical Co Ltd); HLX-06 (Shanghai Henlius Biotech Co Ltd); HNC-VP-L; hVEGF26-104/RFASE (Immunovo BV; Vrije Universiteit van Amsterdam); IBI-302 (Innovent Biologics Inc); imatinib; IMD-0354 (Institute of Medicinal Molecular Design Inc); ITRI-2531 (Industrial Technology Research Institute); K-106 (Kala Pharmaceuticals Inc); KBP-7018 (KBP Biosciences Co Ltd); KD-035 (Jinghua Pharmaceutical Group Co Ltd; Kadmon Pharmaceuticals LLC); KH-902 (Chengdu Kanghong Biotechnologies Co Ltd); KH-902 (Chengdu Kanghong Biotechnologies Co Ltd); KH-903 (Chengdu Kanghong Biotechnologies Co Ltd); KH-906 (Chengdu Kanghong Biotechnologies Co Ltd); KIN-4104 (Kinentia Biosciences LLC); KLH/OPT-821 (MabVax Therapeutics Holdings Inc; Memorial Sloan-Kettering Cancer Center); KN-014 (Suzhou Alphamab Co Ltd); KN-027 (Suzhou Alphamab Co Ltd); L-11885 (National Research Council of Canada); lapatinib; LCB-19 (Interprotein Corp; LegoChem Bioscience Inc); lenvatinib; LMV-12 (Tianjin Longbogen Pharmaceutical Co Ltd); lucitanib; LY-2874455 (Eli Lilly & Co); masitinib; MAT-302 (ElsaLys Biotech); mavrilimumab; metformin; mitothiorole; MP-0250 (Molecular Partners AG); MP-0274 (Molecular Partners AG); muparfostat; necuparanib; nimotuzumab; ningetinib; nintedanib; ODM-203 (Orion Corporation; OMP-305B83 (Celgene Corp; OncoMed Pharmaceuticals Inc); onartuzumab; OSI-930 (Simcere Pharmaceutical Group); OTSGC-A24 (OncoTherapy Science Inc); pacritinib; PAN-90806 (PanOptica Inc); panitumumab; pazopanib; PB (CT)-4010; PPB (CT)-4010 (Paras Biopharmaceuticals Finland Oy); pegaptanib; pegpleranib; perifosine; pertuzumab; PIG-KM (PharmAbcine); PLG-101 (PhiloGene Inc); PLG-201 (PhiloGene Inc); plitidepsin; PMX-20005 (Cellceutix Corp); ponatinib; Pravitinib; PSI-001 (PsiOxus Therapeutics Ltd); PTZ-09 (Shenyang Pharmaceutical University); puquitinib; pyrroltinib; PZ-1 (Synactix Pharmaceuticals Inc); QLNC-3A6 (Qilu Pharmaceutical Co Ltd); ramucirumab; ranibizumab; RAZUMAB (Axxiom Inc; Intas Pharmaceuticals Ltd); rebastinib; recombinant human endostatin; regorafenib; RGX-314 (REGENXBIO Inc); RTEF-651 (Clayton Biotechnologies Inc; Clayton Foundation For Research); RTEF-651 (Clayton Biotechnologies Inc; Clayton Foundation For Research); ruxolitinib; SC-71710 (4SC/Proquinase); SCR-1515 (Simcere Pharmaceutical Group); selinexor; sEphB4-HSA (VasGene Therapeutics); sevacizumab; SIM-010603 (Nanjing Yoko Pharmaceutical Co Ltd; Simcere Pharmaceutical Group); sitravatinib; SKLB-287 (Sichuan University); SL-1026; sorafenib; squalamine; STI-A0168 (Sorrento Therapeutics Inc); sulfatinib; sunitinib; TAB-008 (TOT Biopharm Co Ltd); TOT-102 (TOT Biopharm Co Ltd); TAB-014 (TOT Biopharm Co Ltd; Zhaoke Pharmaceutical (Guangzhou) Co Ltd); TAK-632 (Takeda Pharmaceutical Co Ltd); tanibirumab; TAS-115 (Taiho Pharmaceutical Co Ltd); tepotinib; tesevatinib; tetrathiomolybdate; tivantinib; tivozanib; TJO-054 (Taejoon Pharm Co Ltd); trastuzumab; UB-925 (United BioPharma Inc); UBP-1212 (Shanghai Union Biopharm Co Ltd); UCM-037 (Consejo Superior De Investigaciones Cientificas; Universidad Complutense de Madrid); vandetanib; varlitinib; Vasotide; V-DOS47 (Helix BioPharma Corp); VEGF-165b (University of Bristol); VXM-01 (VAXIMM AG); WS-006 (Waterstone Pharmaceuticals Inc); X-82 (AnewPharma Co Ltd; TyrogeneX); and ZLJ-33 (Institute of Materia Medica Chinese Academy of Medical Sciences & Peking Union Medical College).

In certain embodiments of the present invention, the compound inhibiting the VEGF/VEGFR interaction is selected from the group consisting of 1,2,3-carboxyamido-triazole; ABT-165 (AbbVie Inc); afatinib; aflibercept; Regeneron/Bayer; AL-2846 (Advenchen Laboratories LLC; Chia Tai Tianqing Pharmaceutical Group Co Ltd); albendazole; alferminogene tadenovec; altiratinib; AMC-303 (Amcure GmbH); AN-019; anlotinib; apatinib; AVA-101 (Adverum Biotechnologies Inc; Lions Eye Institute of Western Australia Inc; University of Western Australia); axitinib; BAT-1706 (Bio-Thera Solutions Ltd); bevacizumab; BI-836880 (Boehringer Ingelheim); BMS-817378 (Bristol-Myers Squibb Co; Simcere Pharmaceutical Group); BR-55 (Bracco Research SA); brivanib; cabozantinib; cediranib; cetuximab; CFAK-C10 (CureFAKtor Pharmaceuticals LLC); CFAK-C4 (CureFAKtor Pharmaceuticals LLC); CFAK-C9A (CureFAKtor Pharmaceuticals LLC); cobimetinib; conivaptan; crizotinib; CS-2164 (Shenzhen Chipscreen Biosciences Ltd); dacomitinib; dalantercept; DE-120 (Santen Inc); ENMD-2076 (CASI Pharmaceuticals Inc); erlotinib; famitinib; fenretinide; ficlatuzumab; FP-1039 (Five Prime Therapeutics Inc); fruquintinib; FYB-201 (Bioeq GmbH; Formycon GmbH; Santo Holding AG); galunisertib; gefitinib; ginsenoside Rg3; glesatinib; GNR-011 (Affitech A/S; International Biotechnology Center Generium LLC); HL-217 (Hanlim Pharmaceutical Co Ltd); hVEGF26-104/RFASE (Immunovo BV; Vrije Universiteit van Amsterdam); imatinib; KH-902 (Chengdu Kanghong Biotechnologies Co Ltd); KH-902 (Chengdu Kanghong Biotechnologies Co Ltd); KH-903 (Chengdu Kanghong Biotechnologies Co Ltd); KLH/OPT-821 (MabVax Therapeutics Holdings Inc; Memorial Sloan-Kettering Cancer Center); lapatinib; lenvatinib; lucitanib; LY-2874455 (Eli Lilly & Co); masitinib; mavrilimumab; metformin; MP-0250 (Molecular Partners AG); muparfostat; necuparanib; nimotuzumab; ningetinib; nintedanib; ODM-203 (Orion Corporation; OMP-305B83 (Celgene Corp; OncoMed Pharmaceuticals Inc); onartuzumab; OTSGC-A24 (OncoTherapy Science Inc); pacritinib; PAN-90806 (PanOptica Inc); panitumumab; pazopanib; pegaptanib; pegpleranib; perifosine; pertuzumab; plitidepsin; ponatinib; Pravitinib; puquitinib; pyrroltinib; ramucirumab; ranibizumab; RAZUMAB (Axxiom Inc; Intas Pharmaceuticals Ltd); rebastinib; recombinant human endostatin; regorafenib; ruxolitinib; SC-71710 (4SC/Proquinase); selinexor; sEphB4-HSA (VasGene Therapeutics); sevacizumab; SIM-010603 (Nanjing Yoko Pharmaceutical Co Ltd; Simcere Pharmaceutical Group); sitravatinib; sorafenib; squalamine; sulfatinib; sunitinib; TAB-008 (TOT Biopharm Co Ltd); TOT-102 (TOT Biopharm Co Ltd); tanibirumab; TAS-115 (Taiho Pharmaceutical Co Ltd); tepotinib; tesevatinib; tetrathiomolybdate; tivantinib; tivozanib; trastuzumab; vandetanib; varlitinib; VXM-01 (VAXIMM AG); and X-82 (AnewPharma Co Ltd; TyrogeneX).

In certain embodiments of the present invention, the compound inhibiting the VEGF/VEGFR interaction is selected from the group consisting of afatinib; aflibercept; alferminogene tadenovec; anlotinib; apatinib; axitinib; bevacizumab; brivanib; cabozantinib; cediranib; cetuximab; cobimetinib; conivaptan; crizotinib; dacomitinib; erlotinib; famitinib; fruquintinib; FYB-201 (Bioeq GmbH; Formycon GmbH; Santo Holding AG); galunisertib; gefitinib; ginsenoside Rg3; GNR-011 (Affitech A/S; International Biotechnology Center Generium LLC); imatinib; KH-902 (Chengdu Kanghong Biotechnologies Co Ltd); KH-902 (Chengdu Kanghong Biotechnologies Co Ltd); lapatinib; lenvatinib; masitinib; metformin; muparfostat; nimotuzumab; nintedanib; pacritinib; panitumumab; pazopanib; pegaptanib; pegpleranib; pertuzumab; plitidepsin; ponatinib; pyrroltinib; ramucirumab; ranibizumab; RAZUMAB (Axxiom Inc; Intas Pharmaceuticals Ltd); recombinant human endostatin; regorafenib; ruxolitinib; selinexor; sorafenib; squalamine; sulfatinib; sunitinib; tesevatinib; tivantinib; tivozanib; trastuzumab; and vandetanib

In certain embodiments of the present invention, the compound inhibiting the VEGF/VEGFR interaction is selected from the group consisting of sorafenib, sunitinib, ramucirumab, vatalanib, regorafenib, bevacizumab, brivanib, cabozatinib, cediranib, lenvatinib, linifanib, nintedanib, and ramucirumab.

In certain particular embodiments of the present invention, the compound interfering with the VEGF/VEGFR interaction is sorafenib.

In certain embodiments an HDAC inhibitor is a compound for which an IC₅₀ of 10 μM or lower, or 1 μM or lower, or 500 nM or lower, or 250 nM or lower, or 100 nM or lower, or 50 nM or lower, or 25 nM or lower, or 10 nM or lower, or 5 nM or lower or can be determined for one or more HDAC enzymes selected from the group consisting of HDAC enzymes 1 to 11 in an in vitro assay. Such in vitro assay can for instance be the following assay:

1. Mix assay buffer containing 50 mM Tris-HCl, pH8.0, 137 mM NaCl, 2.7 mM KCl, 1 mM MgCl₂, and add directly before use 1 mg/ml BSA and an acetylated AMC-labeled peptide substrate (RHKKAc for HDAC 1-7 and 9-11; RHKAcKAc for HDAC8) to a final concentration of 50 μM;

2. Add the compound to be analyzed in different predefined final concentrations for each respective data point, dissolved in DMSO to a final concentration of DMSO of 1%);

3. Add the HDAC enzyme (see details and final concentration below);

4. Subsequently, incubate mixture is for 2 hours at 30° C.;

5. Subsequently, add developer (5 mg/mL trypsin/2 μM trichostatin A in the above buffer) in an amount an equal amount of to the above mixture (to stop the reaction and develop the fluorescence signals);

6. Read fluorescence signal (Ex. 355 nm/Em. 460 nm) every 5 min, until signal stabilizes (indicating that trypsin cleavage of the deacetylated peptide substrate to release the AMC fluorophor is complete) (<2 h);

7. Obtain IC₅₀ values and curve fits, e.g. with Graph Pad Software Prism®.

The standard curve can be made from 100 μM compound with 1:2 dilution and 10-doses, 6 μl. The curve can be used to check the HDAC activity each time. Trichostatin A can serve as a control, fluorescent deacetylated standard can be Biomol, Cat # KI-142 (Biomol GmbH, Hamburg, Germany).

Enzymes:

Human HDAC1 (GenBank Accession No. NM_004964): Full length with C-terminal GST tag, MW=79.9 kDa, expressed by baculovirus expression system in Sf9 cells; 75 nM.

Human HDAC2 (GenBank Accession No. Q92769): Full length with C-terminal His tag, MW=60 kDa, expressed by baculovirus expression system in Sf9 cells; 5 nM.

Human HDAC3/NcoR2 (GenBank Accession No. NM_003883 for HDAC3, GenBank Accession No. NM_006312 for NcoR2): Complex of human HDAC3, full length with C-terminal His tag, MW=49.7 kDa, and human NCOR2, N-terminal GST tag, MW=39 kDa, co-expressed in baculovirus expression system; 2.3 nM.

Human HDAC4 (GenBank Accession No. NM_006037): Amino acids 627-1085 with N-terminal GST tag, MW=75.2 kDa, expressed in baculovirus expression system; 266 nM.

Human HDACS (GenBank Accession No. NM_001015053): Full length with Nterminal GST tag, MW=150 kDa, expressed by baculovirus expression system in Sf9 cells; 588 nM; 13 nM.

Human HDAC6 (GenBank Accession No. BC069243): Full length with N-terminal GST tag, MW=159 kDa, expressed by baculovirus expression system in Sf9 cells.

Human HDAC7 (GenBank Accession No. AY302468): Amino acids 518-end with N-terminal GST tag, MW=78 kDa, expressed in baculovirus expression system.

Human HDAC8 (GenBank Accession No. NM018486): Full length, MW=42 kDa, expressed in an E. coli expression system.

Human HDAC9 (GenBank Accession No. NM178423): Amino acids 604-1066 with C-terminal His tag, MW=50.7 kDa, expressed in baculovirus expression system.

Human HDAC10 (GenBank Accession No. NM_032019): Amino acids 1-631 with Nterminal GST tag, MW=96 kDa, expressed by baculovirus expression system in Sf9 cells.

Human HDAC11 (GenBank Accession No. NM_BC009676) with N-terminal GST tag, MW=66 kDa, expressed in baculovirus expression system.

In certain embodiments of the present invention, the HDAC inhibitor is selected from the group consisting of Chidamide, AP-001 (Avenzoar Pharmaceuticals), KA-2507 (Karus Therapeutics), HG-3001 (HitGen), sulforaphane, CG-1255 (Errant Gene Therapeutics), CS-3158 (Shenzhen Chipscreen Biosciences), lovastatin, AR-42 (Arno Therapeutics), VRx-3996 (Viracta Therapeutics), JW-1521 (Errant Gene Therapeutics), CG-200745 (CrystalGenomics), CUDC-907 (Curis), MPT-0E028 (Formosa Laboratories; National Taiwan University; Taipei Medical University), OCID-4681 (BEXEL Pharmaceuticals), QTX-125 (Quimatryx), SP-2528 (Salarius Pharmaceuticals), RG-2833 (BioMarin Pharmaceutical), SF-2558HA (SignalRx Pharmaceuticals), KDAC-001 (kDAC Therapeutics), LB-201 (Lixte Biotechnology), LB-205 (Lixte Biotechnology), bortezomib, thalidomide, romidepsin, ACY-1083 (Acetylon Pharmaceuticals), ACY-257 (Acetylon Pharmaceuticals), ACY-738 (Acetylon Pharmaceuticals; Celgene), citarinostat, 4SC-202 (4SC AG), abexinostat, belinostat, givinostat, panobinostat, pracinostat, sivelestat, tefinostat, ricolinostat, quisinostat, resminostat, tucidinostat, valproic acid, vorinostat, mocetinostat, tosedostat, entinostat, and fidarestat.

In certain embodiments of the present invention, the HDAC inhibitor is selected from the group consisting of Chidamide, 4SC-202 (4SC AG), abexinostat, belinostat, bortezomib, CG-200745 (CrystalGenomics), CUDC-907 (Curis), entinostat, fidarestat, givinostat, lovastatin, mocetinostat, panobinostat, pracinostat, quisinostat, resminostat, ricolinostat, romidepsin, sivelestat, sulforaphane, tefinostat, thalidomide, tosedostat, tucidinostat, valproic acid, vorinostat, and VRx-3996 (Viracta Therapeutics).

In certain embodiments of the present invention, the HDAC inhibitor is selected from the group consisting of 4SC-202 (4SC AG), abexinostat, belinostat, CG-200745 (CrystalGenomics), CUDC-907 (Curis), entinostat, fidarestat, givinostat, mocetinostat, panobinostat, pracinostat, quisinostat, resminostat, ricolinostat, sivelestat, tefinostat, tosedostat, tucidinostat, valproic acid, vorinostat, and VRx-3996 (Viracta Therapeutics).

In certain particular embodiments of the present invention, the compound inhibiting the HDAC inhibitor is resminostat.

As used herein, resminostat (which is an International Non-proprietary Name, i.e. INN) and (E)-3-[1-(4-Dimethylaminomethyl-benzenesulfonyl)-1H-pyrrol-3-yl]-N-hydroxy-acrylamide (its chemical name) are used interchangeably and both refer to a compound of the following formula:

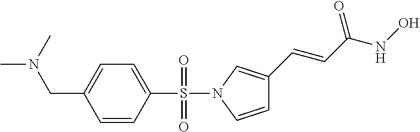 [SMILES: CN(C)Cc1ccc(S(=O)(=O)n2ccc(/C=C/C(=O)NO)c2)cc1]

In particular embodiments of the present invention, said compound inhibiting the VEGF/VEGFR interaction is selected from the group consisting of Sunitinib, Sorafenib, Regorafenib, Ramucirumab and Vatalanib, yet even more particularly Sorafenib.

As used herein, sorafenib (which is an International Non-proprietary Name) and 4-[4-[[4-chloro-3-(trifluoromethyl)phenyl]carbamoylamino]phenoxy]-N-methyl-pyridine-2-carboxamide (its chemical name) are used interchangeably and both refer to a compound of the below formula. Sorafenib is also known under its trade name Nexavar®.

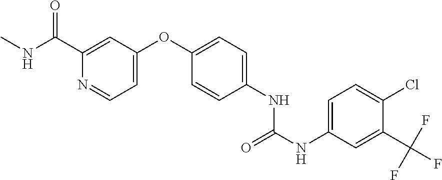 [SMILES: CNC(=O)c1cc(Oc2ccc(NC(=O)Nc3ccc(Cl)c(C(F)(F)F)c3)cc2)ccn1]

In certain embodiments of the present invention, the daily dose of resminostat may be 600 mg, or in other embodiments less than 600 mg, 550 mg or less, 500 mg or less, 500 mg or less, 450 mg or less, 400 mg or less.

In certain specific embodiments, sorafenib is administered at a dose of 800 mg/day, e.g. 400 mg twice daily.

In certain specific embodiments, sorafenib is administered at a dose of 800 mg/day (i.e. 400 mg twice daily) on days 1-14 in a 14 days treatment cycle and Resminostat is administered at a dose of 400 mg/day on days 1-5 in a 14 days treatment cycle.

Suitable salts for resminostat are acid addition salts or salts with bases. Particular mention may be made of the pharmacologically tolerable inorganic and organic acids and bases customarily used in pharmacy. Those suitable are, on the one hand, water-insoluble and, particularly, water-soluble acid addition salts, the acids being employed in salt preparation in an equimolar quantitative ratio or one differing therefrom, particularly in an equimolar quantitative ratio. On the other hand, salts with bases are—depending on substitution—also suitable, the bases being employed in salt preparation in an equimolar quantitative ratio or one differing therefrom. Pharmacologically intolerable salts, which can be obtained, for example, as process products during the preparation of resminostat on an industrial scale, are converted into pharmacologically tolerable salts by processes known to the person skilled in the art. According to the invention, resminostat as well as its salts may contain, e.g. when isolated in crystalline form, varying amounts of solvents. Included within the scope of the invention are therefore all solvates and in particular all hydrates of resminostat as well as all solvates and in particular all hydrates of resminostat, in particular such solvates or hydrates comprising about 0.5, 1 or 2 solvate or water molecules per molecule of resminostat or salts thereof.

Particular salts in the context of the present invention are the salts of resminostat with methanesulfonic acid, in particular in a molar ratio of about 1:1.

Resminostat and salts thereof can be prepared, for example, as described in detail in WO 2005/087724 A2, WO 2007/39404 A1 and WO 2009/112529 A1, respectively.

Sorafenib is commercially available (Nexavar® by Bayer AG) and methods of its preparation are well-known.

The biological and medicinal properties of resminostat and its respective salts, as well as of sorafenib are described in detail in the prior art, including the references cited herein.

In certain embodiments of the present invention, the HDAC inhibitor and the compound inhibiting the VEGF/VEGFR interaction may be administered, simultaneously, sequentially or separately.

In the further context of the present invention the term “active agents” refers to a compound exerting a medical effect on a disease or medical condition (e.g. an amelioration thereof) and said term in particular includes the HDAC inhibitor and the compound inhibiting the VEGF/VEGFR interaction, such as resminostat and sorafenib.

In the embodiments of the invention, the active agents may be provided in pharmaceutical compositions comprising one or more of said active agents and a pharmaceutically acceptable carrier or diluent. In particular, the HDAC inhibitor and the compound inhibiting the VEGF/VEGFR interaction, such as resminostat and sorafenib may be provided in the same pharmaceutical composition (also known as a fixed combination) or in separate pharmaceutical compositions (e.g. in two separate tablets).

Such pharmaceutical compositions may be provided in the context of pharmaceutical products, comprising e.g. one or more pharmaceutical compositions and packaging material. Said packaging material typically comprises a label or package insert which indicates that the active agent(s) is/are useful for treating the diseases detailed herein. The packaging material, label and package insert otherwise parallel or resemble what is generally regarded as standard packaging material, labels and package inserts for pharmaceuticals having related utilities.

The pharmaceutical compositions according to this invention are prepared by processes which are known per se and familiar to the person skilled in the art. As pharmaceutical compositions, the active agents are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions, the active agent content advantageously being between 0.1 and 95% and where, by the appropriate choice of the auxiliaries and/or excipients, a pharmaceutical administration form (e.g. a delayed release form or an enteric form) exactly suited to the active agent and/or to the desired onset of action can be achieved.

The person skilled in the art is familiar with auxiliaries, vehicles, excipients, diluents, carriers or adjuvants which are suitable for the desired pharmaceutical formulations, preparations or compositions on account of his/her expert knowledge. In addition to solvents, gel formers, ointment bases and other excipients, for example antioxidants, dispersants, emulsifiers, preservatives, solubilizers, colorants, complexing agents or permeation promoters, can be used.

Cancer types according to the present invention are in particular ICD-10 type C22.0 hepatocellular carcinoma (HCC, see e.g. https://en.wikipedia.org/wiki/Hepatocellular_carcinoma_and_eMedicine_med/787; http://www.emedicine.com/med/topic787.htm), ICD-10 type C64 renal cell carcinoma (RCC, see e.g. https://en.wikipedia.org/wiki/Renal_cell_carcinoma and eMedicine med/2002; http://www.emedicine.com/med/topic2002.htm), ICD-10 type C73 follicular thyroid cancer (FTC, see e.g. https://en.wikipedia.org/wiki/Follicular_thyroid_cancer and eMedicine med/804; http://www.emedicine.com/med/topic804.htm) or papillary thyroid cancer (PTC, see e.g. https://en.wikipedia.org/wiki/Papillary_thyroid_cancer and eMedicine med/2464; http://www.emedicine.com/med/topic2464.htm), and ICD-10 type C22.1 cholangiocarcinoma (CCA, https://en.wikipedia.org/wiki/Cholangiocarcinoma and eMedicine med/343; http://www.emedicine.com/med/topic343.htm).

Additional therapeutically active agents, which are normally administered to treat cancer, may optionally be administered before or in some cases during treatment with the HDAC inhibitor and the compound inhibiting the VEGF/VEGFR interaction. Examples of such additional therapeutically active agents are known chemotherapeutic anti-cancer agents used in cancer therapy, including, but not are limited to (i) alkylating/carbamylating agents such as Cyclophosphamid (Endoxan®), Ifosfamid (Holoxan®), Thiotepa (ThiotehpaLederle®), Melphalan (Alkeran®), or chloroethylnitrosourea (BCNU); (ii) platinum derivatives like cis-platin (Platinex® BMS), oxaliplatin or carboplatin (Cabroplat® BMS); (iii) antimitotic agents/tubulin inhibitors such as vinca alkaloids (vincristine, vinblastine, vinorelbine), taxanes such as Taxol (Paclitaxel®), Taxotere (Docetaxel®) and analogs as well as new formulations and conjugates thereof; (iv) topoisomerase inhibitors such as anthracyclines (exemplified by Doxorubicin/Adriblastin®), epipodophyllotoxines (examplified by Etoposide/Etopophos®) and camptothecin analogs (exemplified by Topotecan/Hycamtin®); (v) pyrimidine antagonists such as 5-fluorouracil (5-FU), Capecitabine (Xeloda®), Arabinosylcytosine/Cytarabin (Alexan®) or Gemcitabine (Gemzar®); (vi) purin antagonists such as 6-mercaptopurine (Puri-Nethol®), 6-thioguanine or fludarabine (Fludara®) and finally (vii) folic acid antagonists such as methotrexate (Farmitrexat®).

Examples of target specific anti-cancer drug classes used in experimental or standard cancer therapy include but are not limited to (i) kinase inhibitors such as e.g. Glivec (Imatinib®), ZD-1839/Iressa (Gefitinib®), SU11248 (Sutent®) or OSI-774/Tarceva (Erlotinib®); (ii) proteasome inhibitors such as PS-341 (Velcade®); (iii) heat shock protein 90 inhibitors like 17-allylaminogeldanamycin (17-AAG); (iv) vascular targeting agents (VTAs) and anti-angiogenic drugs like the VEGF antibody Avastin (Bevacizumab®) or the KDR tyrosine kinase inhibitor PTK787/ZK222584 (Vatalanib®); (v) monoclonal antibodies such as Herceptin (Trastuzumab®) or MabThera/Rituxan (Rituximab®), mutants as well as conjugates of monoclonal antibodies and antibody fragments; (vi) oligonucleotide based therapeutics like G-3139/Genasense (Oblimersen®); (vii) protease inhibitors (viii) hormonal therapeutics such as anti-estrogens (e.g. Tamoxifen), anti-androgens (e.g. Flutamide or Casodex), LHRH analogs (e.g. Leuprolide, Goserelin or Triptorelin) and aromatase inhibitors.

Other known anti-cancer agents which can be used for combination therapy include bleomycin, retinoids such as all-trans retinoic acid (ATRA), DNA methyltransferase inhibitors such as the 2-deoxycytidine derivative Decitabine (Docagen®), alanosine, cytokines such as interleukin-2, interferons such as interferon α2 or interferon-γ, TRAIL, DR4/5 agonistic antibodies, FasL and TNF-R agonists and finally histone deacetylase inhibitors different to sulphonylpyrrole derivatives as described in the present invention such as SAHA, PXD101, MS275, MGCD0103, Depsipeptide/FK228, NVP-LBH589, valproic acid (VPA) and butyrates.

As exemplary anti-cancer agents for use in combination with the compounds according to this invention in the co-therapies mentioned herein the following drugs may be mentioned, without being restricted thereto, 5 FU, actinomycin D, abarelix, abciximab, aclarubicin, adapalene, alemtuzumab, altretamine, aminoglutethimide, amiprilose, amrubicin, anastrozole, ancitabine, artemisinin, azathioprine, basiliximab, bendamustine, bicalutamide, bleomycin, broxuridine, busulfan, capecitabine, carboplatin, carboquone, carmustine, cetrorelix, chlorambucil, chlormethine, cisplatin, cladribine, clomifene, cyclophosphamide, dacarbazine, daclizumab, dactinomycin, daunorubicin, deslorelin, dexrazoxane, docetaxel, doxifluridine, doxorubicin, droloxifene, drostanolone, edelfosine, eflornithine, emitefur, epirubicin, epitiostanol, eptaplatin, erbitux, estramustine, etoposide, exemestane, fadrozole, finasteride, floxuridine, flucytosine, fludarabine, fluorouracil, flutamide, formestane, foscarnet, fosfestrol, fotemustine, fulvestrant, gefitinib, gemcitabine, glivec, goserelin, gusperimus, herceptin, idarubicin, idoxuridine, ifosfamide, imatinib, improsulfan, infliximab, irinotecan, lanreotide, letrozole, leuprorelin, lobaplatin, lomustine, melphalan, mercaptopurine, methotrexate, meturedepa, miboplatin, mifepristone, miltefosine, mirimostim, mitoguazone, mitolactol, mitomycin, mitoxantrone, mizoribine, motexafin, nartograstim, nebazumab, nedaplatin, nilutamide, nimustine, octreotide, ormeloxifene, oxaliplatin, paclitaxel, palivizumab, pegaspargase, pegfilgrastim, pentetreotide, pentostatin, perfosfamide, piposulfan, pirarubicin, plicamycin, prednimustine, procarbazine, propagermanium, prospidium chloride, raltitrexed, ranimustine, ranpirnase, rasburicase, razoxane, rituximab, rifampicin, ritrosulfan, romurtide, ruboxistaurin, sargramostim, satraplatin, sirolimus, sobuzoxane, spiromustine, streptozocin, tamoxifen, tasonermin, tegafur, temoporfin, temozolomide, teniposide, testolactone, thiotepa, thymalfasin, tiamiprine, topotecan, toremifene, trastuzumab, treosulfan, triaziquone, trimetrexate, triptorelin, trofosfamide, uredepa, valrubicin, verteporfin, vinblastine, vincristine, vindesine, vinorelbine and vorozole.

Other known anti-cancer agents which can be used for combination therapy include agents commonly known as immune checkpoint inhibitors or short checkpoint inhibitors, i.e. agents that inhibit inhibitory checkpoint molecules, such as the inhibitory checkpoint molecules Adenosine A2A receptor (A2AR), B7-H3 (also called CD276), B7-H4 (also called VTCN1), B and T Lymphocyte Attenuator (BTLA, also called CD272), short for Cytotoxic T-Lymphocyte-Associated protein 4 (CTLA-4, also called CD152), Indoleamine 2,3-dioxygenase (IDO), Killer-cell Immunoglobulin-like Receptor (KIR), Lymphocyte Activation Gene-3 (LAG3), Programmed Death 1 receptor (PD-1), as well as Programmed Death 1 receptor Ligand (PD-L1), T-cell Immunoglobulin domain and Mucin domain 3 (TIM-3), V-domain Ig suppressor of T cell activation (VISTA, also called C10orf54). Examples of such checkpoint inhibitors include MGA271 (by MacroGenics), Ipilimumab (Yervoy®), Tremelimumab (formerly CP-675,206), Lirilumab, BMS-986016 (by BMS), BMS-936559/MDX-1105 (by BMS), Pembrolizumab (Keytruda®), Nivolumab (Opdivo®), Galiximab, IMP321 (by Immuntep), BMS-663513 (by BMS), PF-05082566 (by Pfizer), IPH2101 (by Innate Pharma/BMS), KW-0761 (by Kyowa Kirin), CDX-1127 (by CellDex Therapeutics), MEDI-6469 (by MedImmune/AstraZeneca), MEDI4736 (by AstraZeneca), CP-870,893 (by Genentech), Pidilizumab, MPDL3280A (by Genentech), AMP-514 (by MedImmune/AZ), MEDI4736 (by MedImmune/AZ), AUNP 12 peptide (by Aurigene and Pierre Fabre), MSB0010718C (by Merck Serono)

In practicing the present invention and depending on the details, characteristics or purposes of their uses mentioned above, the active agents according to the present invention may be administered in combination therapy separately, sequentially, simultaneously or chronologically staggered (e.g. as combined unit dosage forms, as separate unit dosage forms or adjacent discrete unit dosage forms, as fixed or non-fixed combinations, as kit-of-parts or as admixtures).

A “fixed combination” is defined as a combination wherein a first active ingredient and at least one further active ingredient are present together in one unit dosage or in a single entity. One example of a “fixed combination” is a pharmaceutical composition wherein the said first active ingredient and said further active ingredient are present in admixture for simultaneous administration, such as in a single formulation. Another example of a “fixed combination” is a pharmaceutical combination wherein the said first active ingredient and the said further active ingredient are present in one unit without being in admixture.

A “kit-of-parts” is defined as a combination wherein the said first active ingredient and the said further active ingredient are present in more than one unit. One example of a “kit-of-parts” is a combination wherein the said first active ingredient and the said further active ingredient are present separately. The components of the kit-of-parts may be administered separately, sequentially, simultaneously or chronologically staggered.

The first and further active ingredient of a combination or kit-of-parts according to this invention may be provided as separate formulations (i.e. independently of one another), which are subsequently brought together for simultaneous, sequential, separate or chronologically staggered use in combination therapy; or packaged and presented together as separate components of a combination pack for simultaneous, sequential, separate or chronologically staggered use in combination therapy.

The type of pharmaceutical formulation of the first and further active ingredient of a combination or kit-of-parts according to this invention can be similar, i.e. both ingredients are formulated in separate tablets or capsules, or can be different, i.e. suited for different administration forms, such as e.g. one active ingredient is formulated as tablet or capsule and the other is formulated for e.g. intravenous administration.

A further aspect of the present invention is a combination comprising, in non-fixed form, an HDAC inhibitor such as resminostat or a salt thereof, in particular resminostat mesylate (i.e. methanesulfonate), and one or more art-known standard therapeutic, in particular art-known compound inhibiting the VEGF/VEGFR interaction, such as those mentioned above, in particular sorafenib, for sequential, separate, simultaneous or chronologically staggered use in therapy in any order. Optionally said combination comprises instructions for its use in therapy.

A further aspect of the present invention is a combined preparation, such as e.g. a kit of parts, comprising a preparation of an HDAC inhibitor, such as resminostat or a salt thereof and a pharmaceutically acceptable carrier or diluent; a preparation of a compound inhibiting the VEGF/VEGFR interaction, in particular sorafenib, and a pharmaceutically acceptable carrier or diluent; and optionally instructions for simultaneous, sequential, separate or chronologically staggered use in therapy.

A further aspect of the present invention is a kit of parts comprising a dosage unit of an HDAC inhibitor, such as resminostat or a salt thereof, a dosage unit of a compound inhibiting the VEGF/VEGFR interaction, in particular sorafenib, and optionally instructions for simultaneous, sequential or separate use in therapy.

A further aspect of the present invention is a pharmaceutical product comprising an HDAC inhibitor, such as resminostat, or one or more pharmaceutical compositions comprising said compounds; and a compound inhibiting the VEGF/VEGFR interaction, in particular sorafenib, or one or more pharmaceutical compositions comprising said therapeutic agents, for simultaneous, sequential or separate use in therapy. Optionally this pharmaceutical product comprises instructions for use in said therapy.

A further aspect of the present invention is a pharmaceutical composition as unitary dosage form comprising, in admixture, an HDAC inhibitor, such as resminostat or a salt thereof, a compound inhibiting the VEGF/VEGFR interaction, in particular sorafenib, and optionally a pharmacologically acceptable carrier, diluent or excipient.

A further aspect of the present invention is a commercial package comprising an HDAC inhibitor, such as resminostat or a salt thereof together with instructions for simultaneous, sequential or separate use with a compound inhibiting the VEGF/VEGFR interaction, in particular sorafenib.

In addition, the combination according to the present invention can be used in the pre- or post-surgical treatment.

In further addition, the combination according to the present invention can be used in combination with radiation therapy, in particular in sensitization of patients towards standard radiation therapy.

The administration of the combination according to the present invention and pharmaceutical compositions according to the invention may be performed in any of the generally accepted modes of administration available in the art. Illustrative examples of suitable modes of administration include intravenous, oral, nasal, parenteral, topical, transdermal and rectal delivery. Oral and intravenous delivery are preferred.

In the embodiments of the present invention, doses refer to the amount of compound with respect to the free form of said compound, i.e. the free acid or free base form of said compound. Consequently, adducts, salts, etc. of such free acid or free base form are actually to be administered in a correspondingly higher dose in order to account for the weight of the counter-ion or adduct partner. For example, in relation to resminostat mesylate salt, a “dose of 400 mg resminostat” relates to (rounded) 510 mg resminostat mesylate salt—comprising 400 mg resminostat free base and 110 mg methanesulfonic acid (molecular weight of resminostat=349.4; molecular weight of resminostat mesylate salt=445.5; therefore 400:349.4*445.5=510).

Having described the invention in detail, the scope of the present invention is not limited only to those described characteristics or embodiments. As will be apparent to persons skilled in the art, modifications, analogies, variations, derivations, homologisations and adaptations to the described invention can be made on the base of art-known knowledge and/or, particularly, on the base of the disclosure (e.g. the explicit, implicit or inherent disclosure) of the present invention without departing from the spirit and scope of this invention as defined by the scope of the appended claims.

In the present invention, the administration of active agents may follow a certain schedule, which may include periods of daily administration of active agents and periods wherein no active agents are administered. For example, such a schedule may consist of repeating cycles of 5 days of active agents administration followed by 9 days wherein no active agents are administered (“rest period”) (14-day cycle), 5 days of active agents administration followed by 16 days of rest period (21-day cycle), or 14 days of active agents administration followed by 7 days of rest period (21-day cycle).

### EXAMPLES

The following examples serve to illustrate the invention further without restricting it.

A Phase I/II trial investigating resminostat in the indication of hepatocellular cancer (HCC) was conducted. The study was an open-label, multi-center Phase I/II study in patients with advanced HCC previously untreated with systemic chemotherapy (first-line therapy) to determine the MTD and to evaluate safety and efficacy of resminostat in combination with sorafenib. The study was conducted in Japan and Korea. The main eligibility criteria included a performance status of ECOG 0-1, adequate hepatic, renal, cardiac and bone marrow function and a life expectancy of more than 12 weeks.

In the Phase II part, a total of 170 patients were enrolled. Patients were randomly allocated to either the combination group (86 patients) or the monotherapy group (84 patients) at the RD determined from phase I. Randomization was performed with the minimization method using the following 2 factors as stratification factors: study site; and vascular invasion (presence or absence). The trial is registered at www.clinicaltrials.jp (Identifier: JapicCTI-132124) and www.clinicalTrials.gov (NCT02400788). Overall Survival (OS) were evaluated by the Kaplan-Meier method. A point estimate of the hazard ratio was calculated for each subgroup categorized by baseline platelet level as well as the 95% confidence interval of the estimate.

Treatment regimen: Mono group: A cycle was defined as 14 days. Sorafenib (800 mg) was given daily. Combination group: A cycle was defined as 14 days. Sorafenib (800 mg) was given daily. Resminostat (400 mg) was given daily on Days 1 to 5 and suspended on Days 6 to 14.

Blood was collected into a blood collection tubes precharged with EDTA-2K and then immediately inverted 4-5 times to mix. The mixed sample was stored refrigerated for further analysis.

Platelet count analysis was conducted with the Sheath Flow DC Detection method (Sysmex, Kobe, Japan). A method is described in detail in WALTERS J., GARRITY P. (2000): Performance Evaluation of the Sysmex XE-2100 Hematology Analyzer; Laboratory Hematology 6: 83-92. Platelet count: The Sysmex E200 analyzer was operated in impedance platelet count (PLT-I) mode. For statistical analysis, the software “SAS release 9.3(Windows version)” was used.

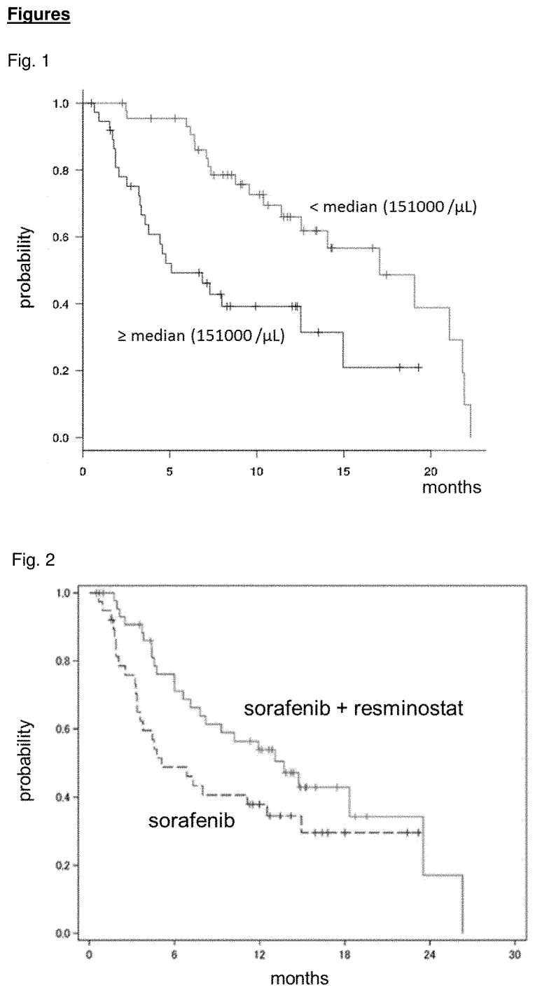

Patients with a platelet count of about 15000 platelets/μL or above had a shorter overall survival (OS) under treatment with sorafenib alone, compared to patients with a platelet count below about 150000 (FIG. 1).

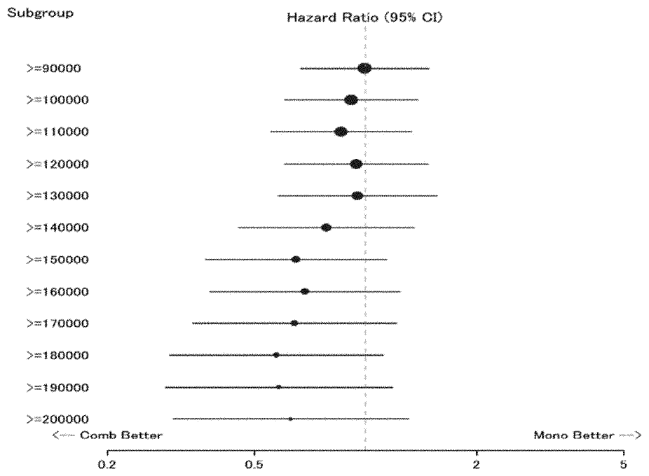

Surprisingly, it was found that patients receiving a combination treatment with the VEGFR/VEGF interaction inhibitor sorafenib and the HDAC inhibitor resminostat showed longer OS if their platelet count was about 140000 platelets/μL or above (FIGS. 2 and 3).

The data analysis yielded the following results (compare FIG. 3):

| | No. of patients | No. of patients in | |
|---|---|---|---|
| Subgroup | in combination | sorafenib mono | Hazard ratio (95% |
| (platelets/μl) | group (%) | group (%) | confidence interval) |
| ≥90000 | 49/81 (60.5) | 49/80 (61.3) | 0.99 (0.67-1.48) |
| ≥100000 | 44/75 (58.7) | 46/76 (60.5) | 0.91 (0.60-1-39) |
| ≥110000 | 41/70 (58.6) | 40/68 (58.8) | 0.86 (0.55-1-34) |
| ≥120000 | 41/68 (60.3) | 37/65 (56.9) | 0.94 (0.60-1.48) |
| ≥130000 | 32/55 (58.2) | 32/58 (55.2) | 0.95 (0.58-1-57) |
| ≥140000 | 26/49 (53.1) | 27/48 (56.3) | 0.78 (0.45-1.36) |
| ≥150000 | 25/46 (54.3) | 25/40 (62.5) | 0.65 (0.37-1.14) |
| ≥160000 | 22/39 (56.4) | 24/38 (63.2) | 0.69 (0.38-1.24) |
| ≥170000 | 20/36 (55.6) | 20/32 (62.5) | 0.64 (0.34-1.22) |
| ≥180000 | 18/32 (56.3) | 19/29 (65.5) | 0.57 (0.29-1.12) |
| ≥190000 | 16/26 (61.5) | 17/26 (65.4) | 0.58 (0.29-1.18) |
| ≥200000 | 15/23 (65.2) | 16/24 (66.7) | 0.63 (0.30-1.31) |

1. A method of treatment of a cancer selected from the group consisting of HCC, RCC, NSCLC, ovarian cancer, CCA, PTC and FTC, said method comprising administering to a subject in need thereof an HDAC inhibitor in combination with a compound inhibiting the VEGF/VEGFR interaction, wherein said subject is characterized in having a platelet count of about 140000 platelets/μl or higher.

2. A composition comprising an HDAC inhibitor for treating cancer selected from the group consisting of HCC, RCC, NSCLC, ovarian cancer, CCA, PTC and FTC in a subject, wherein said composition is to be administered in combination with a compound inhibiting the VEGF/VEGFR interaction, and wherein said subject is characterized in having a platelet count of about 140000 platelets/μl or higher.

3. (canceled)

4. The method according to claim 1, wherein said HDAC inhibitor is resminostat or a salt or solvate thereof.

5. The method according to claim 4, wherein said salt of resminostat is resminostat mesylate salt.

6. The method according to claim 1, wherein said cancer is HCC.

7. The method according to claim 1, wherein said compound inhibiting the VEGF/VEGFR interaction is sorafenib.

8. The method according to claim 1, wherein the platelet count in a blood sample obtainable from said subject is about 150000 platelets/μl or higher.
